(19) United States
(12) Patent Application Publication
(10) Pub. No.: US 2024/0132900 A1
(43) Pub. Date: Apr. 25, 2024

(54) T-DNA Free Gene Editing through Transient Suppressing POLQ in Plants

(71) Applicant: UNIVERSITY OF FLORIDA RESEARCH FOUNDATION, INCORPORATED, Gainesville, FL (US)

(72) Inventors: Heqiang HUO, Gainesville, FL (US); Guiluan WANG, Gainesville, FL (US)

(21) Appl. No.: 18/114,014

(22) Filed: Feb. 24, 2023

Related U.S. Application Data
(60) Provisional application No. 63/313,382, filed on Feb. 24, 2022.

(51) Int. Cl. C12N 15/82; C12N 5/04; C12N 9/22; C12N 15/11; C12Q 1/6895

(52) U.S. Cl. CPC C12N 15/8241; C12N 5/04; C12N 9/22; C12N 15/11; C12N 15/8205; C12Q 1/6895; C12N 2310/20; C12N 2800/80; C12Q 2600/13

(57) ABSTRACT

Disclosed are method and vectors for the application of POLQ in creating T-DNA free gene editing in plants. The method of simultaneous silencing of POLQ during CRISP/CAS9 gene editing increased the T-DNA free gene edited TO tobacco seedlings ratio. The method impacts the application of CRISPR gene editing technology in vegetatively propagated crops such as fruit trees by preventing the introduction of foreign DNA during gene editing.

### BACKGROUND

Genetic modification is essential for plant improvement and fundamental research in plant science (Altpeter et al. 2016). The recent advancement in genome editing has made it one of the most powerful biotechnology tools for plant genetic modification. CRISPR/CAS-mediated genome editing has been widely applied to various plant species for trait improvement (El-Mounadi et al. 2020; Chen et al. 2019; Zhu et al. 2020; Pramanik et al. 2021). The principle of CRISPR/CAS genome editing is based on the DNA double-strand break and non-homologous end-joining (NHEJ) or microhomology-mediated end joining (MMEJ) by the endonucleases-guided RNAs complex at the edited targets, therefore leading to various indels; additionally, insertion of a homologous gene into a specific locus by homologous recombination for gene targeting is another type of genetic modification through CRISPR systems (Atkins and Voytas 2020; Vu et al. 2021; Demirer et al. 2021). The beauty of genome editing compared to conventional approaches for creating genetic variations is that it can precisely modify targets without introducing the unnecessary genomic fragments for undesired DNA modification, which may require a lengthy recurrent selection to eliminate them, thus greatly reducing cost and time for genetic improvement (Chen et al. 2019). However, CRISPR gene editing still relies on the delivery of endonucleases and guided RNAs into plant cells for any genetic modification (Nasti and Voytas 2021; Atkins and Voytas 2020). Currently, Agrobacterium or biolistic mediated transformation are primary methods for delivering CRISPR-Cas reagents in plants. In these scenarios, the CRISPR/Cas will be integrated into genomes, resulting in transgenic plants which are subjected to strict regulation in many countries. Although the transgene can be removed through seed segregations in many annual crops, it will be challenging for trees and clonal plant species with heterosis for desirable traits, in which seed segregation will be hampered by the long juvenile phase and high heterozygosity (Goralogia et al. 2021; Sattar et al. 2021). Alternative approaches were developed for transgene-free gene editing. Exogenous DNA can be removed by molecular excision systems such as Cre/Lox under inducible conditions (Nishihama et al. 2016; Li et al. 2020). DNA-free gene editing can also be achieved by transient expression of CRISPR-Cas components in plants, which require nanoparticles or viral vectors as delivery carriers (Kujur et al. 2021; Demirer et al. 2021). Additionally, ribonucleoprotein (RNP) complex consisting of Cas proteins and guided RNAs are delivered into protoplasts through PEG infiltration, lipofection or electroporation to bypass the application of exogenous DNA integration (Liu et al. 2020; Wu et al. 2020; Woo et al. 2015). Despite that success has been made for generating transgene free gene-edited products with these approaches, great challenges such as inefficiency in reagent delivery, narrow host range or limited cargo capacity, and the inability to regenerate plantlets from the protoplast culture remain for most plant species (Nasti and Voytas 2021, Kujur et al. 2021; Demirer et al. 2021).

### BRIEF DESCRIPTION OF THE DRAWINGS

The present embodiments are illustrated by way of example, and not by way of limitation, in the figures of the accompanying drawings.

The following figures are illustrative only, and are not intended to be limiting.

FIG. 1 shows the engineering vectors for delivery of CRISPR-Cas9 for PDS3 gene mutagenesis. (A) Schematic diagram of the tobacco PDS3 gene showing the target site for CRISPR/Cas9. White and gray boxes indicate coding regions and untranslated regions, respectively. (B) T-DNA constructs of pds3, antiPOLQ/pds3, POLQ-RNAi/pds3 used for targeted gene editing in tobacco. LB, left T-DNA border; RB, right T-DNA border.

FIG. 2 shows the effects of different durations of kanamycin selection on regeneration of T-DNA free mutants. The frequency of T-DNA free pds3 mutants derived from the infection with pds3 and antiPOLQ/pds3 after 3 days, 5 days, 7 days, 9 days and 11 days of 60 mg/L kanamycin treatment were showed. Data are the means±SD of three biological replications with ≥20 samples per replication. Different letters on each bar denote significant differences (P<0.01, ANOVA, Tukey correction).

FIG. 3 shows the effects of different concentrations of kanamycin selection on regeneration of T-DNA free mutants. The frequency of T-DNA free pds3 mutants derived from the infection with pds3 and antiPOLQ/pds3 after 20, 40, 60, 80 and 100 mg/L kanamycin treatment for 7 days was showed. Data are the means±SD of three biological replications with ≥20 samples per replication. Different letters on each bar denote significant differences (P<0.01, ANOVA, Tukey correction).

FIG. 4 shows the dynamic changes of green fluorescence in tobacco callus and shoots during plant regeneration. Green fluorescence was detected and imaged from cotyledons at 3, 5, 7, 9, 11 and 40 days after the infection with antiPOLQ/pds3, POLQ-RNAi/pds3, pds3 and phn102-14 (empty) vectors. 15 explants for each vector were closely tracked, the representative images for each vector were illustrated Images in upper and bottom panels for each construct were photographed under UV and white light. Scale bars, 1 mm.

FIG. 5 shows the improvement of T-DNA free gene editing by transient silencing of POLQ. (A) The frequency of T-DNA free pds3 mutants (chimera and albino) derived from the infection with pds3, antiPOLQ/pds3 and POLQ-RNAi/pds3. (B) Representative PCR verification of T-DNA free pds3 mutant lines (chimera and albino) using GFP (SEQ ID NO. 1-2) and Tubulin primers (SEQ ID NO. 3-4). Tubulin was used as the internal control to examine DNA quality. WT: wild type. Shoots regenerated from the optimized treatment (i.e. 7 days of 60 mg/L Kanamycin selection) were used to extract DNA. (C) The frequency of T-DNA free shoots (albino, chimera and green) derived from the infection with pds3, antiPOLQ/pds3 and POLQ-RNAi/pds3. (D) Representative PCR verification of T-DNA free shoots (albino, chimera and green) using GFP and Tubulin primer. Tubulin was used as the internal control to check DNA quality. WT: wild type. (E) qRT-PCR analysis of the POLQ transcript levels in pds3, antiPOLQ/pds3 and POLQ-RNAi/pds3. 45-day-old shoots with two to three leaves were collected for RNA extraction. At least 60 samples of each mutant or green shoot were tested in B and D. Data in A, C, E are the means±SD of three biological replications with ≥20 samples per replication. Different letters on each bar denote significant differences (P<0.01, ANOVA, Tukey correction).

FIG. 6 shows the editing patterns for CRISPR/Cas9-mediated mutagenesis in the PDS3 gene. (A-C) Mutation patterns identified in the T-DNA-free and T-DNA-integrated mutants from the infection with pds3 (A), antiPOLQ/pds3 (B) and POLQ-RNAi/pds3 (C). The PAM sequences are highlighted in purple. The ‘#’ on the left of the columns indicates the labeled number for different colonies, which corresponds to the sequencing chromatogram of FIG. 8. Deletions and insertions are depicted as dashes and character N, respectively. The number of deleted and/or inserted nucleotides, and their mutation frequency for each sequencing pattern are presented in the right columns; g1: guided RNA1, g2: guided RNA2.

FIG. 7 shows the kanamycin sensitivity of pds3 mutant plants. (A) pds3, antiPOLQ/pds3 and POLQ-RNAi/pds3 shoots were cultured on MS media without kanamycin for 40 days. (B) pds3, antiPOLQ/pds3 and POLQ-RNAi/pds3 shoots were cultured on MS media containing 100 mg/L kanamycin for 40 days. The antiPOLQ/pds3 and POLQ-RNAi/pds3 shoots died, while the pds3 shoot survived from the kanamycin selection.

FIG. 8 shows the representative Sanger sequencing chromatogram of wide type (WT) and the T-DNA integrated or free pds3 mutants from infections with pds3, antiPOLQ/pds3 and POLQ-RNAi/pds3. G1 or G2 in each image are guide RNA1 and guide RNA2. The ‘#’ on the left of the columns indicates the labeled number for different colonies, which corresponds to labelled numbers in FIG. 6.

FIG. 9 provides accession numbers of protein and gene sequences of POLO from a number of plant species.

FIG. 10 provides an identification of components of the sequence of SEQ ID NO: 17.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed through the present specification unless otherwise indicated.

The term “Agrobacterium” refers to a genus of Gram-negative bacteria that uses horizontal gene transfer to cause tumors in plants. Agrobacterium is well known for its ability to transfer DNA between itself and plants. Transformation of plants with Agrobacterium can be achieved in multiple ways. Protoplasts or alternatively leaf-discs can be incubated with the Agrobacterium and whole plants regenerated using plant tissue culture. In agroinfiltration, the Agrobacterium may be injected directly into the leaf tissue of a plant. This method transforms only cells in immediate contact with the bacteria, and results in transient expression of plasmid DNA. Agroinfiltration is commonly used to transform tobacco (Nicotiana). A common transformation protocol is the floral dip method: inflorescence are dipped in a suspension of Agrobacterium, and the bacterium transforms the germline cells that make the female gametes. The seeds can then be screened for antibiotic resistance (or another marker of interest), and plants that have not integrated the plasmid DNA will die when exposed to the correct condition of antibiotic.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

The term “sequence” refers to a nucleotide sequence of any length, which can be DNA or RNA; can be linear, circular or branched and can be either single-stranded or double stranded.

The term “gene of interest” as used herein refers to any nucleotide sequence, which is to be incorporated into the cells to produce genetically transformed cells.

The term “genome editing” refers to modifying a genome with techniques that employ targeted mutagenesis to activate DNA repair pathways. These techniques include, but are not limited to, those that utilize endonucleases to generate single-strand and double-strand DNA breaks that activate DNA repair pathways. Genome editing techniques may also comprise systems that enable targeted editing at any genomic locus. These targeting systems include, but are not limited to, polypeptides, such as, Transcription Activator-Like Effectors (TALEs) and zinc fingers (ZFs), or nucleic acids, such as, Clustered Regularly Interspaced Short Palindromic Repeats/Cas (CRISPR/CAS) single guide RNAs or NgAgo (Argonaute) single strand DNAs. As used herein, “genome editing” and “genome-engineering” are interchangeable. The term “genetic modification” refers to a DNA sequence difference, epigenetic difference, or combination thereof between two genomes of the same species in which one genome is identified as the modified genome and the other is identified as the unmodified genome and the DNA sequence or epigenetic difference is the result of applying genome modifying techniques to the unmodified genome to yield the modified genome. A genetic modification, as used herein, encompasses any insertion, deletion, or substitution of a nucleotide sequence of any size and nucleotide content, any epigenetic modification to any number of nucleotides, or a combination thereof. A genetic modification, as used herein, may also encompass introduction of one or more exogenous coding nucleic acids that do not integrate into the unmodified genome, yet are capable of autonomous replication. In certain embodiments, a modification to an endogenous gene or regulatory element thereof may be a deletion, a substitution, or an insertion that reduces expression of the endogenous gene or the polypeptide for which it encodes. In specific embodiments, the modification may be an indel, wherein the indel may cause a frameshift mutation, a missense mutation, a nonsense mutation, a neutral mutation, or a silent mutation. In specific embodiments, a modification to a regulatory element of an endogenous gene may alter or eliminate a function of the regulatory element. In further contemplated embodiments, the modification may comprise a nucleic acid sequence that provides exogenous control of endogenous gene, mRNA, or polypeptide expression levels. In specific embodiments, the modification may also disrupt a post-translational process of a polypeptide encoded by an endogenous gene. Post-translational processes in certain embodiments may be post-translational modification, protein sorting, or proteasomal degradation.

The term “plant” includes whole plants, plant organs (e.g., leaves, stems, roots, etc.), seeds and plant cells and progeny of same. The class of plants which can be used in the methods of the invention is generally as broad as the class of higher plants amenable to transformation techniques, including both monocotyledonous and dicotyledonous plants. A non-limiting list of plants for which the embodiments described herein may be implemented include monocots, such as, barley, maize, oat, rice, wheat, rye, Sorghum, millet, Tripsacum, Triticale, forage grass and turf grass or dicots such as Berry, Blueberry, Blackberry, Raspberry, Loganberry, Huckleberry, Cranberry, Gooseberry, Elderberry, Currant, Caneberry, Bushberry, Strawberry, Brassica Vegetables, Broccoli, Cabbage, Cauliflower, Brussels Sprouts, Collards, Kale, Mustard Greens, Kohlrabi, Cucurbit Vegetables, Cucumber, Cantaloupe, Melon, Muskmelon, Squash, Watermelon, Pumpkin, Eggplant, Bulb Vegetables, Onion, Garlic, Shallots, Fruiting Vegetables, Pepper, Tomato, Ground Cherry, Tomatillo, Okra, Grape, Herbs/Spices, Leafy Vegetables, Lettuce, Celery, Spinach, Parsley, Radicchio, Legumes/Vegetables (succulent and dried beans and peas), Sunflower, Root/Tuber and Corm Vegetables, Carrot, Potato, Sweet Potato, Cassave, Beets, Ginger, Horseradish, Radish, Ginseng, Turnip, Kiwi, Banana, herbs, Rosemary, Thyme, Cilantro, Oregano, Parsley, Sage, Mint, Lemon grass, Ornamental plants, Annuals, Poinsettia, Helichrysum, Geranium, Begonia, Bacopa, Chrysocephalum, Calibrachoa, Coleus, Cleome, Evolvulus, Angelonia, Argyranthemum, Nemesia, Osteospermum, Petunia, Pansiola, Pelargonium, Cyperus, Dalina, Dahlia, Dichondra, Ipomoea, Lantana, Lobularia, Lobelia, Euphorbia, Impatiens, Verbena, Scaevola, Sedum, Scirpus, Setaceum, Nemesia, Phlox, Torenia, Mecardonia, Perennials, Hydrangea, Clematis, Rosa, Buddleia, Salvia, Sedum, Sambucus, Hybiscus, Weigelia, Hardwood cuttings, Chestnuts, Oak, Maple, Stone Fruit, Apricot, Cherry, Nectarine, Peach, Plum, Prune, Citrus, Orange, Grapefruit, Lemon, Tangerine, Tangelo, or Pummelo.

The term “vector” as used herein refers to a nucleic acid capable of transporting another nucleic acid to which it has been linked. One type of vector is a “plasmid”, which refers to a circular double stranded DNA loop into which additional DNA segments may be ligated. Certain vectors are capable of autonomous replication in a host cell into which they are introduced. In general, expression vectors of utility in recombinant DNA techniques are often in the form of plasmids. In the present specification, “plasmid” and “vector” may be used interchangeably as the plasmid is the most commonly used form of vector.

The terms “POLQ”, “DNA polymerase theta”, or “POLO” refers to a widely conserved DNA polymerase that mediates a microhomology-mediated, error-prone, double strand break (DSB) repair pathway, referred to as Theta Mediated End Joining (TMEJ). Following annealing of short (i.e., a few nucleotides) regions on the DNA overhangs, DNA polymerase theta catalyzes template-dependent DNA synthesis across the broken ends, stabilizing the paired structure.

The term “promoter” refers to a recognition site on a DNA sequence or group of DNA sequences that provides an expression control element for a structural gene and to which RNA polymerase specifically binds and initiates RNA synthesis (transcription) of that gene.

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The term “operably linked” refers to a first nucleic acid sequence connected with a second nucleic acid sequence when the first nucleic acid sequence is placed in a functional relationship with the second nucleic acid sequence. For example, a promoter sequence is “operably linked” to a DNA if the promoter provides for transcription or expression of the DNA. Generally, operably linked DNA sequences are contiguous.

The term “silenc(e), (es) or (ing) the expression” of a gene or polypeptide in a plant or a plant cell includes inhibiting, interrupting, knocking-out, or knocking-down the gene or polypeptide, such that transcription of the gene and/or translation of the encoded polypeptide is reduced as compared to a corresponding control plant, plant cell, or population of plants or plant cells in which expression of the gene or polypeptide is not inhibited, interrupted, knocked-out, or knocked-down. “silencing expression” encompasses any decrease in expression level (e.g., a decrease of 10% or more, 20% or more, 30% or more, 40% or more, 50% or more, 60% or more, 70% or more, 80% or more, 90% or more, or even 100%) as compared to the corresponding control plant, plant cell, or population of plants or plant cells. Expression levels can be measured using methods such as, for example, reverse transcription-polymerase chain reaction (RT-PCR), Northern blotting, dot-blot hybridization, in situ hybridization, nuclear run-on and/or nuclear run-off, RNase protection, or immunological and enzymatic methods such as ELISA, radioimmunoassay, and western blotting.

The term “transformation” or “transform” refers to a process of introducing a DNA sequence or construct (e.g., a vector or expression cassette) into a cell or protoplast in which that exogenous DNA is incorporated into a chromosome or is capable of autonomous replication.

The terms “T-DNA” or “transfer DNA” refer to the transferred DNA of the tumor-inducing (Ti) plasmid of some species of bacteria such as Agrobacterium. The T-DNA is transferred from bacterium into the host plant's nuclear DNA genome. The capability of this specialized tumor-inducing (Ti) plasmid is attributed to two essential regions required for DNA transfer to the host cell. The T-DNA is bordered by 25-base-pair repeats on each end. Transfer is initiated at the right border and terminated at the left border and requires the vir genes of the Ti plasmid. T-DNA is integrated into genomes, resulting in transgenic plants which are subjected to strict regulation in many counties. Although the T-DNA can be eliminated through seed segregations in many annual crops, it will be very challenging for trees and clonal plants with heterosis for desirable traits and requirement of long period for producing progenies due to the long juvenile phase.

The term “construct” or “recombinant construct” (used interchangeably herein) as used herein refers to a construct comprising, inter alia, plasmids or plasmid vectors, cosmids, yeast or bacterial artificial chromosomes (YACs and BACs), phagemid, bacteriophage vectors, an expression cassette, single-stranded or linear nucleic acid sequences or amino acid sequences, and viral vectors, i.e. modified viruses, which can be introduced into a target cell in accordance with the present disclosure. A recombinant construct in accordance with the invention may include CRISPR/Cas tools or parts thereof comprising at least one gRNA or at least one CRISPR nuclease variant and/or at least one further effector domain either in the form of a nucleic acid or an amino acid sequence. Furthermore, the recombinant construct may comprise regulatory sequences and/or localization sequences. The recombinant construct may be integrated into a plasmid vector and/or be isolated from a plasmid vector in the form of a polypeptide sequence or as a single-stranded or double-stranded nucleic acid not linked into a plasmid vector. After introduction, the construct is preferably extrachromosomal and not integrated into the genome and usually in the form of a double-stranded or single-stranded DNA, a double-stranded or single-stranded RNA or a polypeptide. “Plasmid vector,” as used herein, relates to a construct that was originally obtained from a plasmid. These are normally circular, autonomous, replicating, extrachromosomal elements in the form of a double-strand nucleic acid sequence. In genetic engineering, these original plasmids are modified in a targeted manner, in that resistance genes, target nucleic acids, localization sequences, regulating sequences, etc. are inserted. The structural components of the original plasmid, such as the replication source, are maintained thereby. Numerous plasmid vectors for use in a target cell of interest are commercially available, and the modification thereof for specific cloning strategies is well known to the skilled person in the field. These known plasmid vectors are also referred to as standard vectors herein, wherein this is intended to imply that the basis vector is commercially available, and can be readily adapted to the needs of the respective experiment by a skilled person in the corresponding technological field.

The term “vector” or “vector system” as used herein means a transport means which can introduce a recombinant construct, comprising nucleic acids or even polypeptides as well as further sequences such as regulatory sequences or localization sequences directly or indirectly into a desired target cell or target plant structure, into the desired cellular compartment.

The term “vector system” as used here denotes a system which consists of at least one or more vector(s) or contains it(them). Thus, a vector system may comprise a vector which contains/codes for two different recombinant constructs comprising nucleic acid and/or amino acid sequences. Furthermore, a vector system can also contain several vectors which in their turn contain/code for at least one nucleic acid or amino acid sequence in accordance with the present disclosure.

The term “regulatory sequence” as used herein refers to a nucleic acid or a protein sequence which can control cis or trans transcription and/or translation of a disclosed nucleic acid sequence.

The term “regulatory sequence” as used herein refers to a nucleic acid or a protein sequence which can control cis or trans transcription and/or translation of a disclosed nucleic acid sequence.

The term “recombinant” as used herein means a series of nucleic acids or amino acids, in particular not occurring naturally as a totality. Furthermore, the term, “recombinant” also comprises those nucleic acid or amino acid sequencings that occur naturally with regard to their nucleic acid or amino acid sequences, but can also be obtained through a targeted modification or synthesis, e.g. synthetically obtained nucleic acid or amino acid sequences, or through bio-engineering, e.g. nucleic acid or amino acid sequences that are obtained through a fermentative process, which may exist in nature, but can also be produced in a targeted manner in an organism other than the source organism.

“Target region” or “Target site” as used herein refer to any genomic as well as extrachromosomal DNA or RNA, in particular mRNA, of a target organism or a target cell which is to be modified and which can be modified by the method and constructs disclosed herein and is definitely not limited to gene regions, i.e. regions which carry the information for transcription of a mRNA region. These target regions are thus natural or endogenous target regions, wherein the terms, “endogenous” and “natural” are used interchangeably in this context. Moreover, the term, “nucleic acid target region,” is not limited to an endogenous sequence. If an artificial nucleic acid target region has been previously inserted in a target cell of a target structure of interest, the term, “nucleic acid target region,” can thus relate to an artificially inserted nucleic acid target region.

As used herein, the term “fragment” refers to a portion of the nucleic acid sequence. Fragments of sequences useful in transformation methods, using the modified Agrobacterium strains of the disclosure retain the biological activity of the nucleic acid sequence. Alternatively, fragments of a nucleotide sequence that are useful as hybridization probes may not necessarily retain biological activity. Fragments of a nucleotide sequence disclosed herein may range from at least about 20, 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, or 1900, nucleotides, and up to the full length of the subject sequence. A biologically active portion of a nucleotide sequence can be prepared by isolating a portion of the sequence and assessing the activity of the portion.

Fragments and variants of nucleotide sequences and the proteins encoded thereby useful in transformation methods, using the modified Agrobacterium strains of the present disclosure are also encompassed. As used herein, the term “fragment” refers to a portion of a nucleotide sequence and hence the protein encoded thereby or a portion of an amino acid sequence. Fragments of a nucleotide sequence may encode protein fragments that retain the biological activity of the native protein.

Alternatively, fragments of a nucleotide sequence useful as hybridization probes generally do not encode fragment proteins retaining biological activity. Thus, fragments of a nucleotide sequence may range from at least about 20 nucleotides, about 50 nucleotides, about 100 nucleotides, and up to the full-length nucleotide sequence encoding the proteins useful in transformation methods, using the modified Agrobacterium strains of the disclosure.

As used herein, the term “variants” is means sequences having substantial similarity with a sequence disclosed herein. A variant comprises a deletion and/or addition of one or more nucleotides or peptides at one or more internal sites within the native polynucleotide or polypeptide and/or a substitution of one or more nucleotides or peptides at one or more sites in the native polynucleotide or polypeptide. As used herein, a “native” nucleotide or peptide sequence comprises a naturally occurring nucleotide or peptide sequence, respectively. For nucleotide sequences, naturally occurring variants can be identified with the use of well-known molecular biology techniques, such as, for example, with polymerase chain reaction (PCR) and hybridization techniques as outlined herein. A biologically active variant of a protein useful in transformation methods, using the modified Agrobacterium strains of the disclosure may differ from that native protein by as few as 1-15 amino acid residues, as few as 1-10, such as 6-10, as few as 5, as few as 4, 3, 2, or even 1 amino acid residue.

Variant nucleotide sequences also include synthetically derived nucleotide sequences, such as those generated, for example, by using site-directed mutagenesis. Generally, variants of a nucleotide sequence disclosed herein will have at least 40%, 50%, 60%, 65%, 70%, 75%, 80%, 85%, 90%, 91%, 92%, 93%, 94%, to 95%, 96%, 97%, 98%, 99% or more sequence identity to that nucleotide sequence as determined by sequence alignment programs described elsewhere herein using default parameters. Biologically active variants of a nucleotide sequence disclosed herein are also encompassed. Biological activity may be measured by using techniques such as Northern blot analysis, reporter activity measurements taken from transcriptional fusions, and the like. See, for example, Sambrook, et al., (1989) Molecular Cloning: A Laboratory Manual (2d ed., Cold Spring Harbor Laboratory Press, Cold Spring Harbor, N.Y.), hereinafter “Sambrook”, herein incorporated by reference in its entirety. Alternatively, levels of a reporter gene such as green fluorescent protein (GFP) or yellow fluorescent protein (YFP) or the like produced under the control of a promoter operably linked to a nucleotide fragment or variant can be measured. See, for example, Matz et al. (1999) Nature Biotechnology 17:969-973; U.S. Pat. No. 6,072,050, herein incorporated by reference in its entirety; Nagai, et al., (2002) Nature Biotechnology 20(1):87-90. Variant nucleotide sequences also encompass sequences derived from a mutagenic and recombinogenic procedure such as DNA shuffling. With such a procedure, one or more different nucleotide sequences can be manipulated to create a new nucleotide sequence. In this manner, libraries of recombinant polynucleotides are generated from a population of related sequence polynucleotides comprising sequence regions that have substantial sequence identity and can be homologously recombined in vitro or in vivo. Strategies for such DNA shuffling are known in the art. See, for example, Stemmer, (1994) Proc. Natl. Acad. Sci. USA 91:10747-10751; Stemmer, (1994) Nature 370:389 391; Crameri, et al., (1997) Nature Biotech. 15:436-438; Moore, et al., (1997) J. Mol. Biol. 272:336-347; Zhang, et al., (1997) Proc. Natl. Acad. Sci. USA 94:4504-4509; Crameri, et al., (1998) Nature 391:288-291 and U.S. Pat. Nos. 5,605,793 and 5,837,458, herein incorporated by reference in their entirety.

Methods for mutagenesis and nucleotide sequence alterations are well known in the art. See, for example, Kunkel, (1985) Proc. Natl. Acad. Sci. USA 82:488-492; Kunkel, et al., (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein, herein incorporated by reference in their entirety. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

The nucleotide sequences of the disclosure can be used to isolate corresponding sequences from other organisms, particularly other plants, more particularly other monocots or dicots. In this manner, methods such as PCR, hybridization and the like can be used to identify such sequences based on their sequence homology to the sequences set forth herein. Sequences isolated based on their sequence identity to the entire sequences set forth herein or to fragments thereof are encompassed by the present disclosure.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in, Sambrook, supra. See also, Innis, et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York), herein incorporated by reference in their entirety. Known methods of PCR include, but are not limited to, methods using paired primers, nested primers, single specific primers, degenerate primers, gene-specific primers, vector-specific primers, partially-mismatched primers and the like.

In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides and may be labeled with a detectable group such as 32P or any other detectable marker. Thus, for example, probes for hybridization can be made by labeling synthetic oligonucleotides based on the sequences of the disclosure. Methods for preparation of probes for hybridization and for construction of genomic libraries are generally known in the art and are disclosed in Sambrook, supra.

In general, sequences that have activity and hybridize to the sequences disclosed herein will be at least 40% to 50% homologous, about 60%, 70%, 80%, 85%, 90%, 95% to 98% homologous or more with the disclosed sequences. That is, the sequence similarity of sequences may range, sharing at least about 40% to 50%, about 60% to 70%, and about 80%, 85%, 90%, 95% to 98% sequence similarity.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller, (1988) CABIOS 4:11-17; the algorithm of Smith, et al., (1981) Adv. Appl. Math. 2:482; the algorithm of Needleman and Wunsch, (1970) J. Mol. Biol. 48:443-453; the algorithm of Pearson and Lipman, (1988) Proc. Natl. Acad. Sci. 85:2444-2448; the algorithm of Karlin and Altschul, (1990) Proc. Natl. Acad. Sci. USA 872:264, modified as in Karlin and Altschul, (1993) Proc. Natl. Acad. Sci. USA 90:5873-5877, herein incorporated by reference in their entirety. Computer implementations of these mathematical algorithms are well known in the art and can be utilized for comparison of sequences to determine sequence identity.

As used herein, “sequence identity” or “identity” in the context of two nucleic acid or polypeptide sequences refers to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically, this involves scoring a conservative substitution as a partial rather than a full mismatch, thereby increasing the percentage sequence identity. Thus, for example, where an identical amino acid is given a score of one and a non-conservative substitution is given a score of zero, a conservative substitution is given a score between zero and one. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

### DETAILED DESCRIPTION

It was found that suppressing POLQ activity in plants increases the frequency of T-DNA free gene-edited mutants. Agrobacterium were transformed with a vector containing a CRISPR-Cas9 for PDS3 gene mutagenesis and antiPOLQ or POLQ-RNAi cassettes for suppressing POLQ. Tobacco plants were infected with the transformed Agrobacterium and cultivated under various selection conditions and different durations to optimize transfection. The optimum transfection conditions were found to culturing the plants for 7 to 9 days in culture media with a kanamycin concentration of 60 mg/L. The frequency of T-DNA free mutants (chimera and albino) for plant with suppressed POLQ activity reached 47.8% and 44.5%, whereas only 20.6% of control plants were T-DNA free. Additionally, the method described here also significantly increased the frequency of T-DNA free homozygous edited mutants. For example, transient silencing of POLQ in antiPOLQ/pds3, and POLQ-RNAi/pds3 led to 71.1% and 64.2% T-DNA free homozygous albino mutant, whereas 28.6%, and 24.6% of chimeric mutants from antiPOLQ/pds3, and POLQ-RNAi/pds3 are T-DNA free. On the other hand, the T-DNA free chimeric or homozygous albino mutants for the control pds3 vector are only 10.7% and 30%, respectively. These results suggest that the suppressing of POLQ particularly increase the frequency of T-DNA free homozygous edited mutants.

Disclosed are vectors for T-DNA free gene editing in plants and methods of use thereof. In some embodiments, the vector is constructed with sequences that code for oligonucleotides for silencing POLQ and while simultaneous transiently expressing a gene editing system in a plant cell.

### Overview

Agrobacterium tumefaciens-mediated transformation is the most widely used method for generating transgenic plants. The transgene referred to herein as T-DNA is flanked by the two repeated sequences, Left and Right Border repeats. These sequences are recognized and nicked by the virulence proteins VirD1 and VirD2, resulting in a single-strand form of T-DNA. The VirD2 protein remains a covalent link with the single-stranded T-DNA, and guides it to enter the nucleus. The single-stranded T-strands are subsequently converted to double-stranded molecules in the nucleus, resulting in transient expression of transgene prior to random integration of T-DNA into the host genome (transient transformation) (Gelvin 2021). The T-DNA integration was recently found to be dependent on polymerase theta (Pol θ). DNA polymerase theta (Polk), encoded by POLQ) is the predominant mediator of microhomology-mediated end-joining (MMEJ) (also called alternative end-joining) for double-strand break (DSB) repair in most Eukaryotes (Ramsden et al. 2021; Shen et al. 2017; Brambati et al. 2020).

CRISPR-Cas endonucleases introduce DSB that can be repaired by different mechanisms, including error-prone classical non-homologous end-joining (cNHEJ), MMEJ, homology-directed repair (HDR), leading to various mutations that is fundamental for CRISPR genome editing (Manghwar et al. 2019; Brambati et al. 2020; Ramsden et al. 2021). Given the important role of POLQ in MMEJ, defect in POLQ may affect genome editing efficiency. In this disclosure, RNAi silencing has been employed to suppress POLQ to increase the T-DNA-free gene editing events. Deficiency in Polk) did not affect the transient transformation, particularly when a higher concentration of Agrobacterium was used for infection, suggesting that the T-DNA was successfully transferred into plant cells for transient transformation but failed to be integrated into the plant genome for stable transformation (Nishihama et al. 2016; van Kregten et al. 2016). The transient expression of plant cells was sufficient for gene editing by CRISPR/CAS9, as proved by RNP methods in multiple plant species (Zhang et al. 2021). However, the overall gene editing was slightly reduced by silencing of POLQ in the study (Tables 1 and 2). This result is consistent with the observation in the moss Physcomitrella patens. When CRISPR/CAS9 nuclease and small guide RNAs were transiently transformed into protoplasts of wild-type moss and its polq mutant, the mutation frequency was somewhat lower in polq moss mutant (Mara et al. 2019).

Additionally, van Tol et al. (2021) have shown that frequency of gene targeting (knock-in) through CRISPR/Cas9 was reduced in Polθ-deficient Arabidopsis plants, and that gene targeting events obtained in teb (polq)mutant background lacked additional T-DNA copies. Likewise, the POLQ inactivation suppressed random integration of exogenous target gene into the genome of alga species Chlamydomonas reinhardtii through CRISPR/Cas9 (Sizova et al. 2021). The role of POLQ in exogenous DNA integration has also been reported in mammalian cells (Zelensky et al. 2017; Saito et al. 2017), corroborating its conserved role in error-prone DSB repair for genome stability.

With the advent of CRISPR genome editing, the application of this cutting-edge technology has dramatically increased in the genetic improvement of crops. However, the stable transformation of CRISPR/CAS-sgRNA complex is generally needed to achieve a higher frequency of gene editing in plants, resulting in edited products containing transgenes that may concern the general public and are strictly regulated by different legislation. Several strategies have been developed to prevent or remove transgenes, and each one has its disadvantages and advantages.

Genetic segregation is predominantly used for eliminating transgene in seeded annual plant species. The transgene in the host plant genome can be easily removed by screening the progenies segregated from TO transgenic plants. This method is relatively simple and easy with the assistance of PCR detection for transgene presence. To minimize the effort for detecting the transgene-free progenies, screening methods were advanced by simultaneous induction of other phenotypic markers along with the gene-editing cassettes. Although genetic segregation has been successfully employed to eliminate the transgene in many seeded crops, one or more generations may be required for achieving this goal. More importantly, it is difficult to apply this strategy for plants with long juvenile phases such as apple. Genetic segregation to remove transgene is not effective in plants primarily with asexual production modes, which produce identical somatic embryos or vegetative clones.

The methods in this disclosure have advantages over the old methods, particularly for non-seeded perennial plants. The regular Agrobacterium transformation protocol developed for each species is used in this method, which does not have technical challenges for RNP method. Regeneration from protoplasts for RNP has been established in a limited number of plant species, whereas regeneration from regular explants such as cotyledons can be achieved in many plant species. Compared to the particle bombardment that causes a high frequency of random integration of DNAs with various sizes and detectable genomic damage, Agrobacterium-mediated DNA delivery result in much clean genomic background. In addition, the application of selection pressure at the early stage of tissue culture in our method could reduce the wild-type seedling by delaying their growth, thus minimizing the later efforts for screening and identifying gene-edited plants. The disclosed method is a user-friendly and more efficient approach for DNA-free gene editing in many plant species compared to other current approaches.

### Vector Components

In certain embodiments, the vector of the disclosure may include expression cassettes which include the necessary genetic elements for gene editing in plants without the inclusion of T-DNA. In one embodiment, the vector is constructed with sequences that code for oligonucleotides for silencing POLQ. The oligonucleotides for silencing are in a separate expression cassette from the gene editing system. In certain embodiment, the oligonucleotides are RNAi or antisense oligonucleotides. The vector is constructed with one or both oligonucleotides, and the oligonucleotides are operable linked to a promoter region. In a specific embodiment, the promoter region is CaMV35S for facilitating high level of RNA transcription in a wide variety of plants. The oligonucleotides are transiently expressed in the plants.

The vector comprises an expression cassette which contains a sequence for a gene editing system. That cassette comprises, operably joined, a transcriptional initiation region functional in a plant genome, at least one heterologous DNA sequence coding for a gene editing system and control sequences positioned upstream for the 5′ and downstream from the 3′ ends and a transcription termination region to provide transient expression of the gene editing system in the target plant. Preferably, the expression cassette is flanked by plant DNA sequences, in order to facilitate transient expression of the vector in the plant. In the construction of the expression cassette, the DNA sequence comprises one or more cloning site(s) for integration of the gene editing system.

As is known, it will be generally advisable to have at least one additional heterologous nucleotide sequence coding for a selectable phenotype, such as a gene providing for antibiotic resistance or a functional portion thereof to serve as a selection marker associated with the expression cassette or with the universal integration expression vector. This facilitates identification of the cells in which the foreign gene has been stably integrated. Selectable marker genes are known in the literature, for instance aminoglycoside phosphotransferase, kanamycin and neomycin resistant genes such as NPTII, the gene which encodes aminoglycoside phosphotransferase. In some embodiments, the selectable marker comprises GFP.

### Genetic Transformation Methods

The disclosure provides a gene editing method which can produce a gene edited plant free of T-DNA. The method for gene editing a plant uses the vector constructed with oligonucleotides for silencing POLQ. Any method of gene editing of the plant may be used. Any gene editing system which may be utilized to transform a plant genome is suitable for transformation with the vector. The methods of gene editing disclosed utilizes Agrobacterium to edit the genome of the plant cells. In a specific embodiment, the Agrobacterium is Agrobacterium tumefaciens. The Agrobacterium are transformed with the vector. In some embodiments, the Agrobacterium are transformed using heat and thaw methods.

In some embodiments, genome engineering of a plant cell as described herein may employ RNA-guided endonucleases including, but not limited to CRISPR/Cas systems. CRISPR/Cas systems have been described in U.S. Patent Application Publication Nos. 2017/0191082 and 2017/0106025, each of which are incorporated herein by reference in their entirety. In some embodiments, a targeted genome modification as described herein comprises the use of at least one, at least two, at least three, at least four, at least five, at least six, at least seven, at least eight, at least nine, or at least ten RNA-guided nucleases. In some embodiments, a CRISPR/Cas9 system, a CRISPR/Cas12a system, a CRISPR/CasX system, or a CRISPR/CasY system are alternatives that may be used to generate modifications to target sequences as described herein.

The CRISPR systems are based on RNA-guided endonucleases that use complementary base pairing to recognize DNA sequences at target sites. CRISPR/Cas systems are part of the adaptive immune system of bacteria and archaea, protecting them against invading DNA, such as viral DNA, by cleaving the foreign DNA in a sequence-dependent manner. The immunity is acquired by the integration of short fragments of the invading DNA known as spacers between two adjacent repeats at the proximal end of a CRISPR locus. The CRISPR arrays, including the spacers, are transcribed during subsequent encounters with invasive DNA and are processed into small interfering CRISPR RNAs (crRNAs) approximately 40 nt in length, which combine with the trans-activating CRISPR RNA (tracrRNA) to activate and guide the Cas9 nuclease. This cleaves homologous double-stranded DNA sequences known as protospacers in the invading DNA.

A prerequisite for cleavage is the presence of a conserved protospacer-adjacent motif (PAM) downstream of the target DNA, which usually has the sequence 5′-NGG-3′ but less frequently NAG. Specificity is provided by the so-called “seed sequence” approximately 12 bases upstream of the PAM, which must match between the RNA and target DNA. Cpfl acts in a similar manner to Cas9, but Cas12a does not require a tracrRNA. Specificity of the CRISPR/Cas system is based on an RNA-guide that use complementary base pairing to recognize target DNA sequences. In some embodiments, the site-specific genome modification enzyme is a CRISPR/Cas system. In an aspect, a site-specific genome modification enzyme provided herein can comprise any RNA-guided Cas endonuclease (non-limiting examples of RNA-guided nucleases include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpfl, homologs thereof, or modified versions thereof); and, optionally, the guide RNA necessary for targeting the respective nucleases.

In some embodiments, an RNA-guided endonuclease is the DNA cleavage domain of a restriction enzyme fused to a deactivated Cas9 (dCas9), for example dCas9-Fok1. As used herein, a “dCas9” refers to an endonuclease protein with one or more amino acid mutations that result in a Cas9 protein without endonuclease activity, but retaining RNA-guided site-specific DNA binding. As used herein, a “dCas9-restriction enzyme fusion protein” is a dCas9 with a protein fused to the dCas9 in such a manner that the restriction enzyme is catalytically active on the DNA.

In one aspect, a method and/or composition provided herein comprises one or more, two or more, three or more, four or more, or five or more guide RNAs or DNAs. In another aspect, a CRISPR/CAS system, dCas9-restriction enzyme fusion protein, provided herein is capable of generating a targeted DSB in a target sequence as described herein. In one aspect, vectors comprising nucleic acids encoding one or more, two or more, three or more, four or more, or five or more guide RNAs or DNAs and the corresponding CRISPR/CAS system or dCas9-restriction enzyme fusion protein are provided to a cell by transformation methods known in the art (e.g., without being limiting, viral transfection, particle bombardment, PEG-mediated protoplast transfection or Agrobacterium-mediated transformation).

The plasmid vector comprising the target-specific oligonucleotides can then be used for transformation of a plant. The gene constructs can also be introduced by the Agrobacterium-mediated transient and stable transformation.

Transformed plant cells which are produced by any of the above transformation techniques can be cultured to regenerate a whole plant which possesses the transformed genotype and thus the desired phenotype. Such regeneration techniques rely on manipulation of certain phytohormones in a tissue culture growth medium, typically relying on a biocide and/or herbicide marker or reporter marker genes (such as GFP or GUS gene) which have been introduced together with the desired nucleotide sequences. Plant regeneration from cultured protoplasts is described in Evans, et al., “Protoplasts Isolation and Culture” in Handbook of Plant Cell Culture, pp. 124-176, Macmillian Publishing Company, New York, 1983; and Binding, Regeneration of Plants, Plant Protoplasts, pp. 21-73, CRC Press, Boca Raton, 1985. Regeneration can also be obtained from plant callus, explants, organs, pollens, embryos or parts thereof. Such regeneration techniques are described generally in Klee et al (1987) Ann. Rev. of Plant Phys. 38:467-486.

### Antisense and RNA Interference

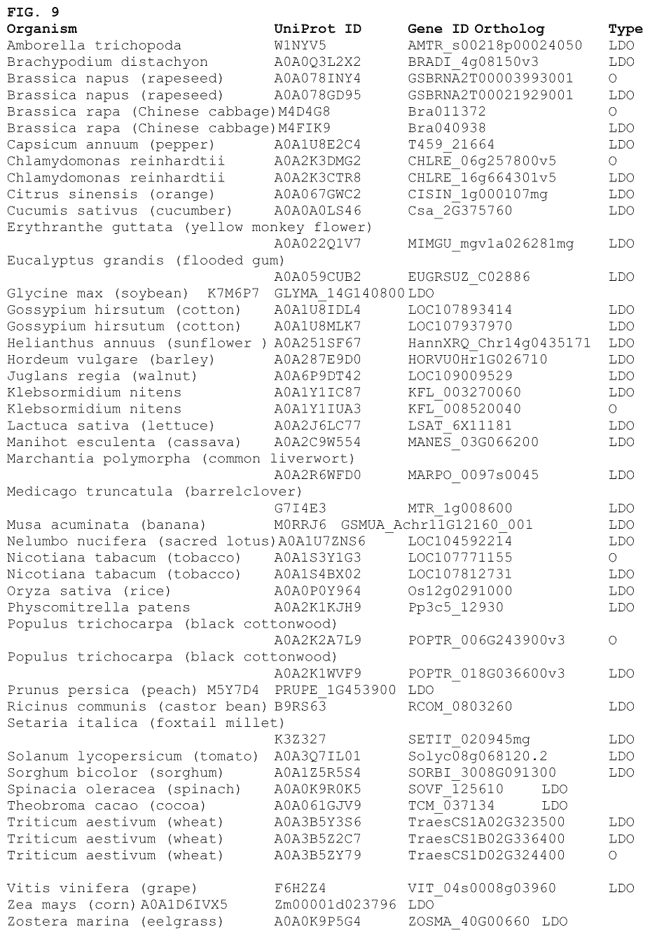

The Examples describe use of Agrobacterium that have been modified to produce POLQ antisense and RNAi molecules for tobacco. Those skilled in the art, in light of the teachings herein, would appreciate that POLQ silencing molecules can be generated for a target plant species and introduced to Agrobacterium such as through a plasmid vector or other suitable means. For example, FIG. 9 provides accession numbers of POLQ sequences for a number of species. One skilled in the art knowing the sequences of the POLQ gene of a target plant could produce oligonucleotides having homology or complementarity to such POLQ genes. Typically, the oligonucleotides would have from 10-400, from 18-300 or 21-300 bp having a sequence that comprises at least 18 contiguous nucleotides to the POLQ gene or RNA transcript thereof. In specific examples, the silencing oligonucleotide comprises at least 80%, 90%, 95% or 98% sequence identity to a fragment of the POLQ gene sequence or RNA transcript thereof. In other embodiments, the oligonucleotide comprises a sequence having 18-29 bp, including all integers in between, with 100% identity or 100% complementarity to a POLQ gene sequence or RNA transcript thereof. The expression of the oligonucleotides in the Agrobacterium can be transferred to the target plant and serve to silence the POLQ in a plant cell of the target plant. A number of oligonucleotides can be produced based on the target POLQ sequence, or obtained from publicly available sources, and tested for silencing effect according to methods known in the art.

### EXAMPLES

### Example 1. Materials and Methods

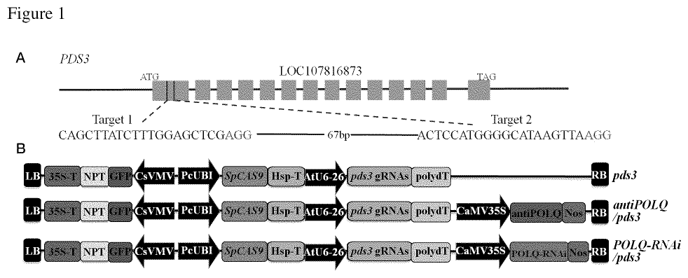

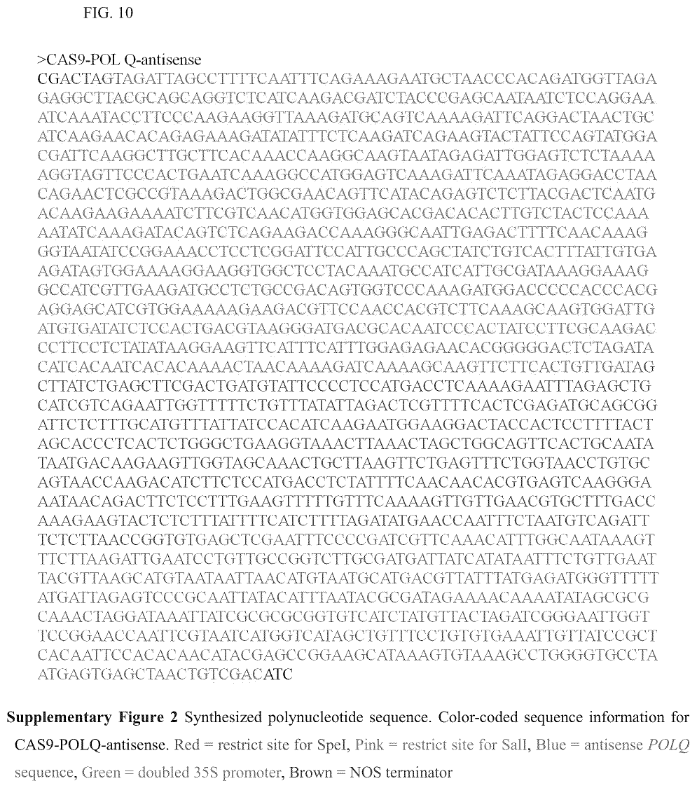

Plant materials and growth conditions. Nicotiana tabacum cv. Samsun wild-type (WT) were grown in a growth chamber at 25° C. with a 16-h light/8-h dark photoperiod. Seeds were germinated on MS media supplemented with 30 g/L sucrose. 10-days-old green seedlings were then infected with Agrobacterium.
Vector construction. A previously constructed plasmid PHN-SpCas9-4x BsaI-GFP (phn102) was used for all following modifications (Nguyen et al. 2021). This plasmid contains an eGFP-NPTII fused protein gene driven by a CsVMV (Cassava vein mosaic virus) promoter for dual selection and an Arabidopsis codon-optimized SpCas9 driven by Parsley Ubiquitin promoter, and an Arabidopsis thaliana U6-26 gene promoter for expressing guided RNAs (Nguyen et al. 2021). Two pairs of primer (NtPDS3-CAS9-F1/R1 and NtPDS3-CAS9-F2/R2) (SEQ ID NO. 5-8) for two guided RNAs to target tobacco PDS3 gene (LOC107816873) were designed and annealed to ligate the plasmid at four BsaI restriction sites to create pds3 vector (FIG. 1). A fragment containing reverse complementary POLQ (LOC104108784) driven by a CaMV35S and terminated by NOS terminator was synthesized by Gene Universal Inc (Newark, DE, USA), and inserted into the pds3 vector through digestion and ligation with SpeI and SalI restriction enzymes to create antiPOLQ/pds3 vector (SEQ ID NO. 17). For the POLQ-RNAi/pds3 vector, the primer set of pRNAi-POLQ-F/R (SEQ ID NO. 9-10) was used to amplify the sense and antisense fragments and cloned to pRNAi vector according to the previously described method (Yan et al. 2012). The POLQ-RNAi fragments were amplified with the primer set (POLQ RNAi-Cas9-F/R)(SEQ ID NO. 11-12) and ligated into the pds3 vector at SpeI and SalI sites to form POLQ-RNAi/pds3 vector (FIG. 1). SEQ ID NO: 19, the antisense sequence for POLQ, represents bp 914-1193 from SEQ ID NO: 17 (see also FIG. 10) (SEQ ID NO: 19) gcttatc tgagcttcga ctgatgtatt cccctccatg acctcaaaagaatttagagc tgcatcgtca gaattggttt ttctgtttat attagactcg ttttcactcgagatgcagcg gattctcttt gcatgtttat tatccacatc aagaatggaa ggactaccactccttttact agcaccctca ctctgggctg aaggtaaact taaactagct ggcagttcactgcaatataa tgacaagaag ttggtagcaa actgcttaag ttctgagttt ctggtaacctgtgcagtaac caagacatct tctccatgac ctctattttc aacaacacgt gagtcaagggaaataacaga cttctccttt gaagtttttg tttcaaaagt tgttgaacgt gctttgaccaaagaagtact ctctttattt tcatctttta gatatgaacc aatttctaat gtcagatttctcttaaccgg tgt
Tobacco transformation. The pds3, antiPOLQ/pds3 (SEQ ID NO: 17) and POLQ-RNAi/pds3 (SEQ ID NO: 18) plasmids were introduced into Agrobacterium tumefaciens strain EHA105 using the freeze and thaw method. Leaf explants were cut from 7-10 day old tobacco seedlings and wounded with razors for Agrobacterium infection. The wounded leaf discs were infected with EHA105 Agrobacterium for 10 minutes and then incubated in the dark at 25° C. for 2 days on the callus induction medium (CIM) containing MS salts, 30 mg/L sucrose, 1.0 mg/L benzylaminopurine, and 0.1 mg/L NAA before being transferred to CIM supplemented with 100 mg/L Timentin (to repress Agrobacterium growth) and different concentrations of Kanamycin (20, 40, 60, 80, 100 mg/L). The infected explants were cultured under the light for different durations (3d, 5d, 7d, 9d, 11d). After antibiotic selection, leaf discs were transferred to shoot induction medium (SIM) supplemented with 100 mg/L Timentin. In the case of tobacco, the hormone and nutrient composition of SIM are same as CIM except the supplement of different antibiotics in the SIM. The CIM and SIM for other plant species should be accordingly adjust based on the medium recipes used for that specific species. The visual screening was performed to identify albino gene-edited mutant shoots.
Fluorescence imaging. GFP fluorescence in Agrobacterium-infected leaves was monitored with a Leica fluorescence microscope (Leica, Germany)
Detection of gene-edited mutations. Genomic DNA was extracted from regenerated shoots with a CTAB method. PCR was performed with a specific primer set (NtPDS3-CAS9-PCR-F/R) (SEQ ID NO. 13-14) to amplify genomic fragments containing the target sites. To confirm the editing of each line and to identify mutant genotypes, PCR amplicons were cloned into the pGEM-T Easy vector (Promega, USA). Varied numbers of colonies for each line were randomly selected for Sanger sequencing with the primer set (NtPDS3-CAS9-sequencing-F/R). Sequencing results were analyzed with Synthego Performance Analysis (https://ice.synthego.com) for detecting edited mutations.
Quantitative real-time PCR analysis. Total RNA was extracted using RNAzol reagent (RN190) (Molecular Research Center, USA) and treated with TURBO DNase (AM2238) (Thermo Fisher Scientific, USA). First-strand cDNA was synthesized with QuantiTect Reverse Transcription Kit (Qiagen, USA). RT-qPCR was performed using the Bio-Rad CFX96 qPCR instrument (Bio-Rad, USA). Each reaction contained 1×iTaq Universal SYBR Green Supermix (Bio-Rad, USA), 300 nM each of POLQ-specific primers (qRT-POLQ F/R SEQ ID NO. 15-16) or Tubulin-specific primers (qRT-α-Tubulin-F/R) and 5% diluted first-strand cDNA. Tubulin was used as the internal control. Three biological replicates and two technical replicates were applied to each sample. Sequences of the primers used in qRT-PCR are listed in sequence ID listing. The transcript levels of genes were calculated by the relative quantitation method (2 (Livak and Schmittgen 2001).
Determination of non-transgenic mutant plants. Stable integration of the CRISPR/Cas9 T-DNA was determined by PCR detection of GFP presence in the tobacco genomic DNA. GFP primers (GFP detector-F/R SEQ ID NO. 1-2) were used to amplify an 886-bp region of the T-DNA fragment. Tubulin was used as the internal control. The absence of GFP PCR fragment was considered the lack of stable transgene integration into the tobacco genome, and mutants were categorized as T-DNA free mutants. Shoots from pds3 mutant plants were also cultured on MS media containing 100 mg/L kanamycin for 40 days to examine kanamycin resistance for T-DNA free validation.

### Example 2. Effect of Different Durations of Antibiotic Selection on T-DNA Free Gene Editing

To test whether simultaneous silencing of POLQ during genetic transformation will prevent T-DNA integration to improve the frequency of T-DNA free gene-edited products, pCaMV35S::antiPOLQ or pCaMV35S::POLQ-RNAi were inserted into a binary expression vector that contains an Arabidopsis optimized SpCas9 driven by a Parsley Ubiquitin promoter and two guided RNAs driven by Arabidopsis U6-26 promoter for targeting PDS3 gene in tetraploid tobacco (Nicotiana tabacum cv. Samsun) (FIG. 1). In addition, all expression vectors also have an eGFP-NPTII fusion gene under a double cassava vein virus promoter for dual selections (FIG. 1B) (Nguyen et al. 2021; Fauser et al. 2014).

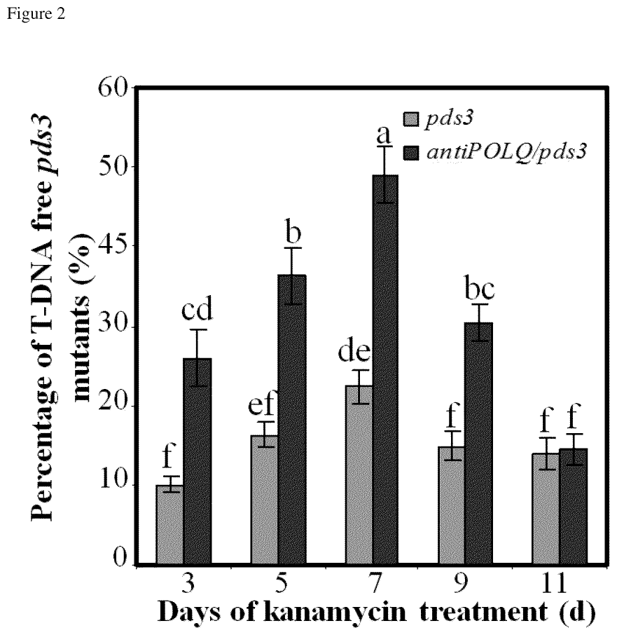

Previous reports showed that T-DNA could be transferred into cell nuclei, and transient expression of T-DNA can still occur (Nishizawa-Yokoi et al. 2021; Gelvin 2021; van Kregten et al. 2016). Therefore, it was hypothesized that T-DNA's transient expression would confer cells with antibiotic resistance until these proteins are degraded. A short selection duration for antibiotic resistance would promote preferential growth of cells with the transiently expressed transgene, but prolonged exposure of cells to antibiotic selection will limit the T-DNA free gene-edited cells. Thus, the optimal duration of antibiotic selection was examined for generating T-DNA free gene-edited shoots with the antiPOLQ/pds3 and pds3 expression vector (FIG. 1). After co-culture on callus induction media (CIM) without selection antibiotics for two days, the infected explants were cultured on a CIM with 60 mg/L of Kanamycin for 3, 5, 7, 9, and 11 days before being transferred to shoot induction medium (SIM) with no antibiotics (FIG. 2 and Table 1). It was observed that the overall mutation frequency (chimeric and albino seedlings/total seedlings) reached the highest for both control pds3 and the antiPOLQ/pds3 vectors as selection duration prolonged to 7 and 9 days (Table 1). In addition, the overall mutation efficiency is higher for control pds3 vector than the antiPOLQ/pds3 vector across all five antibiotic selection treatments (Table 1). However, the ratios of albino seedlings from antiPOLQ/pds3 infection are much higher at 7 and 9 days of antibiotic selection treatments. For example, 10 out of 22 (10 albino+12 chimera) or 45.5%, and 9 out of 15 (9 albino+6 chimera) or 60% were albino seedlings for antiPOLQ/pds3 vector at 7 and 9 days of antibiotic selection, respectively. By contrast, 7 out of 25 (7 albino+18 chimera) or 28%, and 3 out of 18 (3 albino+15 chimera) or 16.7% albino seedlings were generated for pds3 control vector at 7 and 9 days of antibiotic selection, respectively (Table 1). This result might indicate that the mutants from antiPOLQ/pds3 infection could be derived from the earlier gene-editing events that led to homozygous mutations.

TABLE 1
Effects of different durations of kanamycin selection on gene editing
efficiency for the control pds3 and antiPOLQ/pds3 constructs.

| | pds3 | | | | | antiPOLQ/pds3 | | | | |
|---|---|---|---|---|---|---|---|---|---|---|
| | Number | Number of | | | Mutation | Number | Number of | | | Mutation |
| | of | regenerated shoots | | | efficiency | of | regenerated shoots | | | efficiency |
| Days¹ | explants | Green | Chimera | Albino | (%)² | explants | Green | Chimera | Albino | (%)² |
| 3 d | 62 | 198 | 10 | 16 | 11.61 | 49 | 197 | 6 | 8 | 6.64 |
| 5 d | 53 | 133 | 12 | 16 | 17.39 | 49 | 123 | 4 | 8 | 8.89 |
| 7 d | 35 | 95 | 18 | 7 | 20.49 | 67 | 144 | 12 | 10 | 13.25 |
| 9 d | 35 | 93 | 15 | 3 | 16.22 | 41 | 108 | 6 | 9 | 12.20 |
| 11 d | 33 | 94 | 6 | 10 | 14.55 | 41 | 112 | 10 | 5 | 11.81 |

¹The days of kanamycin treatment. The concentration for all treatments was 60 mg/L.
²The mutation efficiency was calculated as follows: (number of chimera + number of albino) ÷ number of regenerated shoots × 100

PCR genotyping results revealed that a much higher frequency of T-DNA free mutants was obtained with antiPOLQ/pds3 vector compared to the control pds3 vector (FIG. 2). For example, 26%, 36.3%, 49% and 30.5% of gene-edited mutants with antibiotic selection for 3, 5, 7, 9 days are T-DNA free for antiPOLQ/pds3 vector, whereas 10%, 16.3%, 22.3% and 14.9% for the control pds3 vector under the same treatments (FIG. 2). By contrast, no significant difference was observed for the frequency of T-DNA free mutants obtained through antiPOLQ/pds3 vector and the control pds3 vector for 11 days. These results showed that transient expression of T-DNA may confer tobacco cells with the best antibiotics resistance for 7-9 days, and prolonged exposure to antibiotics will inhibit the growth of cells with edited targets for antiPOLQ/pds3.

### Example 3. Effect of Different Concentrations of Antibiotic Selection on T-DNA Free Gene Editing

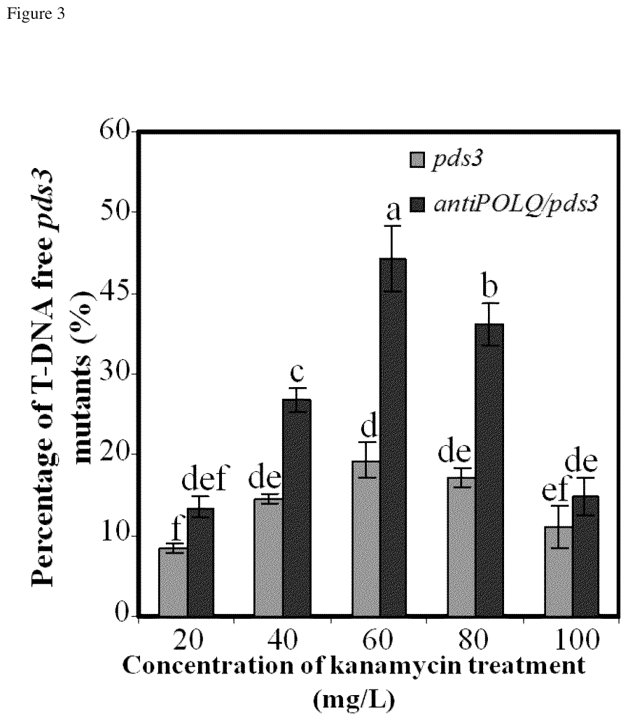

It was asked if a better mutation efficiency and higher frequency of T-DNA free gene-edited mutants will be obtained when different concentrations of antibiotics were applied for the transient selection. Five different concentrations of Kanamycin (20, 40, 60, 80, 100 mg/L) were tested for seven days of selection. As the concentration of applied Kanamycin increased, the mutation efficiency increased (Table 2). The mutation efficiency for both antiPOLQ/pds3 and pds3 control vectors was the highest with 100 mg/L Kanamycin (Table 2), whereas fewer albino plantlets were obtained when 20 and 40 mg/L Kanamycin were applied for both antiPOLQ/pds3 and pds3 control vectors (Table 2). In addition, the mutation efficiency with antiPOLQ/pds3 is lower than the ones with pds3 control vector for all five concentrations of Kanamycin treatments. Despite the highest mutation frequency observed for the treatment with 100 mg/L Kanamycin, the percentage of chimeric mutants (25 out of 33 or 75.8%) is much higher than that with 60 and 80 mg/L treatments (10 out of 20 or 50%, 16 out of 26 or 61.5% respectively) for the antiPOLQ/pds3 (Table 2). PCR genotyping results revealed that the highest frequency of T-DNA free mutants was obtained with 60 mg/L treatments for both antiPOLQ/pds3 and pds3 control vectors (FIG. 3). Therefore, combined with mutation efficiency, 60 mg/L was selected as the concentration of antibiotic selection for subsequent experiments.

TABLE 2
Effects of different concentrations of kanamycin selection on gene editing
efficiency for the control pds3 and antiPOLQ/pds3 constructs.

| | pds3 | | | | | antiPOLO/pds3 | | | | |
|---|---|---|---|---|---|---|---|---|---|---|
| | Number | Number of | | | Mutation | Number | Number of | | | Mutation |
| | of | regenerated shoots | | | efficiency | of | regenerated shoots | | | efficiency |
| Kan¹ | explants | Green | Chimera | Albino | (%)² | cxplants | Green | Chimera | Albino | (%)² |
| 20 | 48 | 133 | 6 | 8 | 9.52 | 48 | 143 | 4 | 0 | 2.72 |
| 40 | 46 | 128 | 10 | 6 | 11.11 | 48 | 142 | 8 | 0 | 5.33 |
| 60 | 50 | 149 | 23 | 11 | 18.58 | 48 | 157 | 10 | 10 | 11.30 |
| 80 | 50 | 145 | 20 | 17 | 20.33 | 48 | 141 | 16 | 10 | 15.57 |
| 100 | 46 | 102 | 22 | 17 | 27.66 | 48 | 115 | 25 | 8 | 22.30 |

¹Concentration of kanamycin treatment (mg/L). The selection duration for all treatments was 7 days.
²The mutation efficiency was calculated as follows: (number of chimera + number of albino) ÷ number of regenerated shoots × 100

### Example 4. Gene Editing by Transient Expression of CAS9

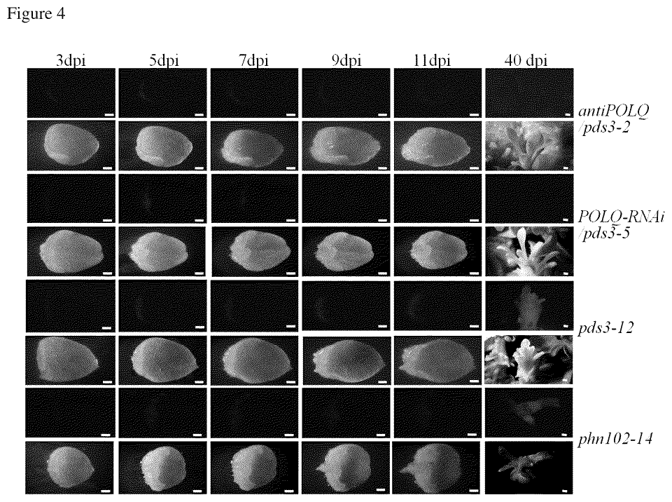

To better understand the mutations created by the transient expression of Cas9, the transient expression of T-DNA gene (GFP) in infected leaf discs was observed at 7 days of 60 mg/L Kanamycin selection with a fluorescent microscope (FIG. 4). Strong GFP fluorescence was detected in tobacco leaf discs 3, 5, and 7 days post infections (dpi) with antiPOLQ/pds3, POLQ-RNAi/pds3, pds3 (pds3-12) vectors or the empty control vectors (phn102-14 with no guided RNAs). However, the GFP fluorescent signals quickly decayed at 9 and 11 days of infections with antiPOLQ/pds3 and POLQ-RNAi/pds3, but increased in the cases of tissues infected with pds3 or the empty control vectors (FIG. 4). Furthermore, although albino mutants were generated from these tracked explants, GFP fluorescence were predominately detected in the mutants from infection with the pds3 vector, whereas a large fraction of mutants from infections with antiPOLQ/pds3 and POLQ-RNAi/pds3 lack detectable GFP fluorescence (FIG. 4). As expected, no albino mutant was generated from the infection with empty vector, albeit strong GFP fluorescence present in the shoots. These results demonstrated that the transient expression of CAS9 was sufficient for editing genes and that the application of GFP may facilitate preliminary selection of T-DNA free shoots to minimize the downstream screening effort.

### Example 5. Improvement of T-DNA Free Gene Editing by Transient Silencing of POLO

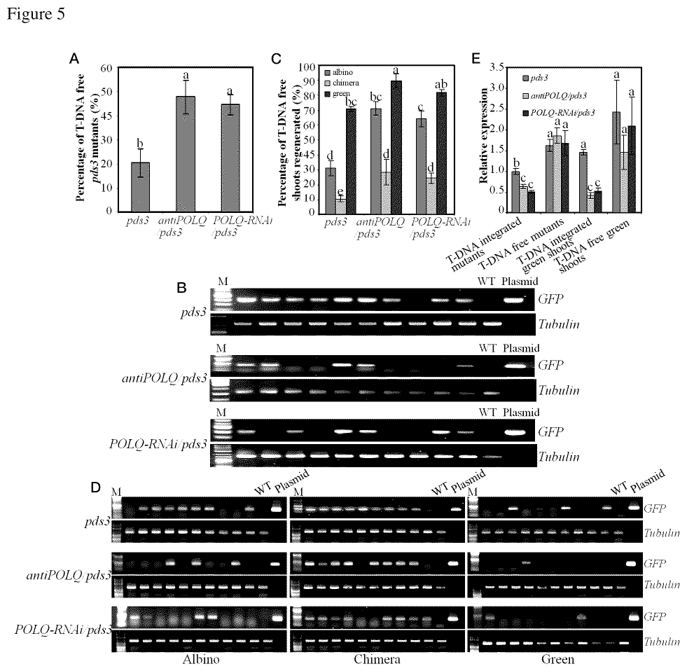

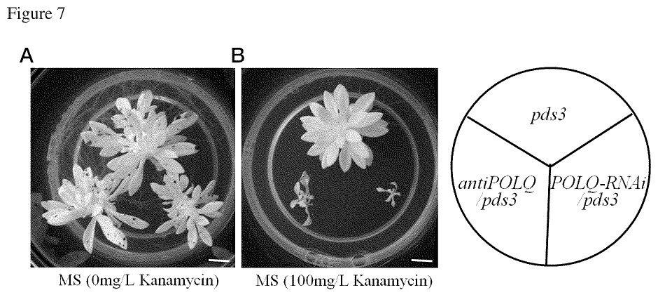

With the optimized treatment conditions, the frequency of T-DNA free gene-edited mutants generated from the infection was compared with control pds3, antiPOLQ/pds3, and POLQ-RNAi/pds3. The frequency of T-DNA free mutants (chimera and albino) for antiPOLQ/pds3 and POLQ-RNAi/pds3 reached 47.8% and 44.5%, whereas only 20.6% of chimera and albino mutants from pds3 vector infection are T-DNA free (FIG. 5A). Furthermore, molecular genotyping for the presence of GFP and antibiotic resistance test of mutants confirmed the presence or absence of T-DNA (FIG. 5B and FIG. 7).

Next, the effect of silencing POLQ on T-DNA insertion was characterized in three different types of shoots (green, chimera, and albino). As shown in FIGS. 5C and 5D, 70.8%, 89.7%, and 81.8% of green shoots did not contain T-DNA for pds3, antiPOLQ/pds3, and POLQ-RNAi/pds3, respectively, indicating that majority of de novo green shoots were not successfully transformed when no antibiotics was applied during shoot regeneration. Interestingly, only 10.7%, 28.6%, and 24.6% of chimeric mutants derived from pds3, antiPOLQ/pds3, and POLQ-RNAi/pds3 did not have T-DNA (FIGS. 5C and D). By contrast, transient silencing of POLQ in antiPOLQ/pds3, and POLQ-RNAi/pds3 led to 71.1% and 64.2% T-DNA free albino mutant, and even ˜30% albino mutants generated with pds3 vector are non-transgenic. These results suggested that editing events may occur at a very early stage in the callus cells, resulting in homozygous albino mutants. On the other hand, the chimeric mutants were mainly attributed to the de novo editing events at a late stage in the stably transformed shoot (FIGS. 5C and D).

To examine whether silencing POLQ is responsible for the regeneration of T-DNA free gene-edited shoots, qRT-PCR was performed to check the POLQ transcript level in the regenerated shoots at 45 days after Agrobacterium infections. For expression analysis in regenerated shoots, T-DNA integrated mutants, T-DNA free mutants, T-DNA integrated green shoots and T-DNA free green shoots were pooled for three different vectors (pds3, antiPOLQ/pds3, and POLQ-RNAi/pds3). As expected, there is no significant difference in the POLQ transcript level in all T-DNA free mutants regardless of their sources (pds3, antiPOLQ/pds3, and POLQ-RNAi/pds3); neither is in T-DNA free green shoots (FIG. 5E). However, the transcript level in the transgenic mutants or green shoots from infection with antiPOLQ/pds3, and POLQ-RNAi/pds3 was lower compared to that in transgenic plants derived from infection with pds3 vector, suggesting that POLQ in these transgenic shoots was partially silenced by antiPOLQ/pds3 or POLQ-RNAi/pds3 (FIG. 5E).

### Example 6. Effect of Silencing POLQ on Gene Editing Pattern in Pds3 Mutants

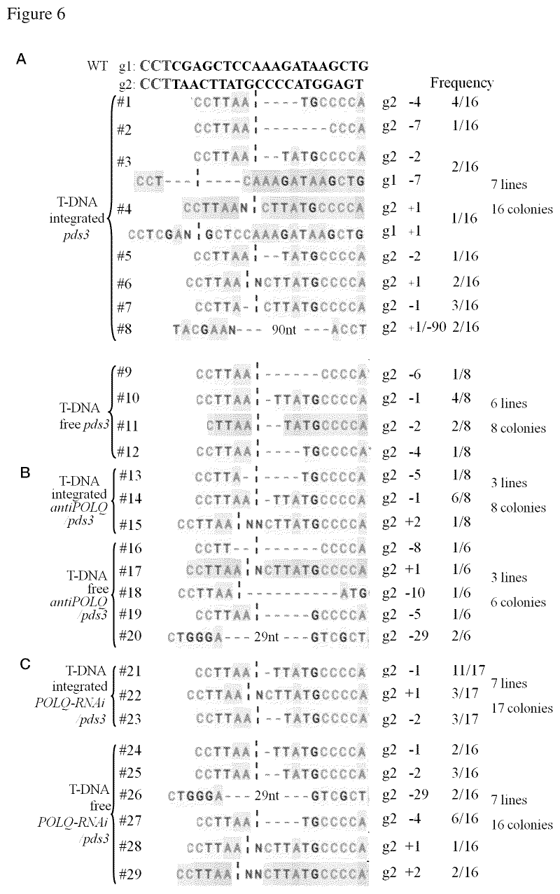

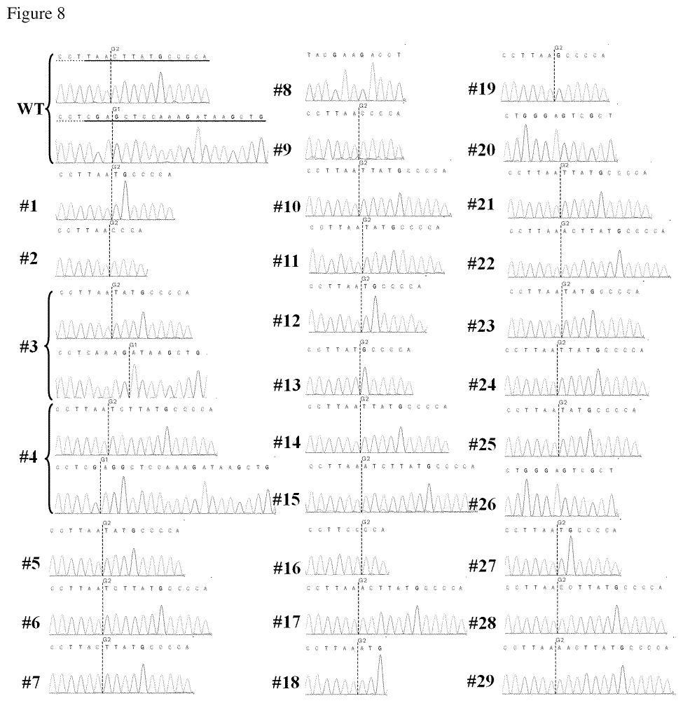

Silencing POLQ increased the percentage of T-DNA free albino mutants, which promoted the examination of whether silencing POLQ affected editing pattern in pds3 mutants. Therefore, the amplified fragments spanning two target loci were subcloned for Sanger sequencing (FIG. 6 and FIG. 8). Overall, 16 colonies from 7 transgenic mutants and 8 colonies from 6 T-DNA free mutants derived from pds3 vector infection, 8 colonies from 3 transgenic mutants and 6 colonies from 3 T-DNA free mutants regenerated from antiPOLQ/pds3 infection, 17 colonies from 7 transgenic mutants, and 16 colonies from 7 T-DNA free mutants derived from POLQ-RNAi/pds3 infection were subjected to Sanger sequencing. It was observed that large deletion editing was slightly more frequent in non-transgenic mutants regenerated from infections with antiPOLQ/pds3, and POLQ-RNAi/pds3. For example, a 29 nt deletion was detected in two colonies from one or two independent mutants regenerated from infection with antiPOLQ/pds3 or POLQ-RNAi/pds3; in addition, a 8 nt deletion and 10 nt deletion were also detected in two colonies from T-DNA free mutant regenerated from infection with antiPOLQ/pds3, and a 4 nt deletion was detected in 6 colonies from two T-DNA free mutants derived from infection with POLQ-RNAi/pds3. By contrast, the editing mutation in transgenic plants regenerated from infections with antiPOLQ/pds3 or POLQ-RNAi/pds3 are predominantly 1 bp deletions (FIGS. 6B and C). On the other hand, large deletion seemed more frequent in transgenic mutant regenerated from infection with pds3 vector; for example, there are 90 bp deletions in 2 colonies from one T-DNA integrated mutants.

### REFERENCES

Altpeter F, Springer N M, Bartley L E, Blechl A E, Brutnell T P, Citovsky V, Conrad U, Gelvin S B, Jackson D P, Kausch A P, Lemaux P G, Medford J I, Orozco-Cardenas M L, Tricoli D M, Van Eck J, Voytas D F, Walbot V, Wang K, Zhang Z Y J, Stewart C N (2016) Advancing Crop Transformation in the Era of Genome Editing. Plant Cell 28 (7):1510-1520. doi:10.1105/tpc.16.00196
Atkins P A, Voytas D F (2020a) Overcoming bottlenecks in plant gene editing. Curr Opin Plant Biol 54:79-84. doi:10.1016/j.pbi.2020.01.002
Brambati A, Barry R M, Sfeir A (2020) DNA polymerase theta (Pol theta)—an error-prone polymerase necessary for genome stability. Curr Opin Genet Dev 60:119-126. doi:10.1016/j.gde.2020.02.017
Chen K L, Wang Y P, Zhang R, Zhang H W, Gao C X (2019) CRISPR/Cas Genome Editing and Precision Plant Breeding in Agriculture. Annu Rev Plant Biol 70:667-697. doi:10.1146/annurev-arplant-050718-100049
Chen L Z, Li W, Katin-Grazzini L, Ding J, Gu X B, Li Y J, Gu T T, Wang R, Lin X C, Deng Z N, McAvoy R J, Gmitter F G, Deng Z A, Zhao Y D, Li Y (2018) A method for the production and expedient screening of CRISPR/Cas9-mediated non-transgenic mutant plants. Hortic Res-England 5. doi:ARTN 1310.1038/s41438-018-0023-4
Demirer G S, Silva T N, Jackson C T, Thomas J B, D W E, Rhee S Y, Mortimer J C, Landry M P (2021) Nanotechnology to advance CRISPR-Cas genetic engineering of plants. Nat Nanotechnol 16 (3):243-250. doi:10.1038/s41565-021-00854-y
El-Mounadi K, Morales-Floriano M L, Garcia-Ruiz H (2020) Principles, Applications, and Biosafety of Plant Genome Editing Using CRISPR-Cas9. Front Plant Sci 11. doi:ARTN 5610.3389/fpls.2020.00056
Fauser F, Schiml S, Puchta H (2014) Both CRISPR/Cas-based nucleases and nickases can be used efficiently for genome engineering in Arabidopsis thaliana. Plant Journal 79 (2):348-359. doi:10.1111/tpj.12554
Gelvin S B (2010) Plant Proteins Involved in Agrobacterium-Mediated Genetic Transformation. Annu Rev Phytopathol 48:45-68. doi:10.1146/annurev-phyto-080508-081852
Gelvin S B (2017) Integration of Agrobacterium T-DNA into the Plant Genome. Annu Rev Genet 51:195-217. doi:10.1146/annurev-genet-120215-035320
Gelvin S B (2021) Plant DNA Repair and Agrobacterium T-DNA Integration. Int J Mol Sci 22 (16). doi:ARTN 845810.3390/ijms22168458
Ghogare R, Ludwig Y, Bueno G M, Slamet-Loedin I H, Dhingra A (2021) Genome editing reagent delivery in plants. Transgenic Res 30 (4):321-335. doi:10.1007/s11248-021-00239-w
Goralogia G S, Redick T P, Strauss S H (2021) Gene editing in tree and clonal crops: progress and challenges. In Vitro Cell Dev-Pl 57 (4):683-699. doi:10.1007/s11627-021-10197-x
He Y B, Zhu M, Wang L H, Wu J H, Wang Q Y, Wang R C, Zhao Y D (2018) Programmed Self-Elimination of the CRISPR/Cas9 Construct Greatly Accelerates the Isolation of Edited and Transgene-Free Rice Plants. Molecular Plant 11 (9):1210-1213. doi:10.1016/j.molp.2018.05.005
He Y B, Zhu M, Wang L H, Wu J H, Wang Q Y, Wang R C, Zhao Y D (2019) Improvements of TKC Technology Accelerate Isolation of Transgene-Free CRISPR/Cas9-Edited Rice Plants. Rice Sci 26 (2):109-117. doi:10.1016/j.rsci.2018.11.001
Hwang H H, Yu M, Lai E M (2017) Agrobacterium-mediated plant transformation: biology and applications. Arabidopsis Book 15:e0186. doi:10.1199/tab.0186
Khakhar A, Voytas D F (2021) RNA Viral Vectors for Accelerating Plant Synthetic Biology. Front Plant Sci 12. doi:ARTN 66858010.3389/fpls.2021.668580
Kralemann L, de Pater S, Shen H, Kloet S, van Schendel R, Hooykaas PJJ, Tijsterman M (2022) T-DNA integration in plants requires MRE11- or TDP2-mediated removal of the 5′ bound Agrobacterium protein VirD2. Research Square. doi:doi.org/10.21203/rs.3.rs-1144888/v1
Kujur S, Senthil-Kumar M, Kumar R (2021) Plant viral vectors: expanding the possibilities of precise gene editing in plant genomes. Plant Cell Reports 40 (6):931-934. doi:10.1007/s00299-021-02697-2
Li J, Li Y, Pawlik K M, Napierala J S, Napierala M (2020) A CRISPR-Cas9, Cre-lox, and Flp-FRT Cascade Strategy for the Precise and Efficient Integration of Exogenous DNA into Cellular Genomes. CRISPR J 3 (6):470-486. doi:10.1089/crispr.2020.0042
Liu W S, Rudis M R, Cheplick M H, Millwood R J, Yang J P, Ondzighi-Assoume C A, Montgomery G A, Burris K P, Mazarei M, Chesnut J D, Stewart C N (2020)
Lipofection-mediated genome editing using DNA-free delivery of the Cas9/gRNA ribonucleoprotein into plant cells. Plant Cell Reports 39 (2):245-257. doi:10.1007/s00299-019-02488-w
Livak K J, Schmittgen T D (2001) Analysis of relative gene expression data using real-time quantitative PCR and the 2(T)(−Delta Delta C) method. Methods 25 (4):402-408. doi:10.1006/meth.2001.1262
Lu H P, Liu S M, Xu S L, Chen W Y, Zhou X, Tan Y Y, Huang J Z, Shu Q Y (2017) CRISPR-S: an active interference element for a rapid and inexpensive selection of genome-edited, transgene-free rice plants. Plant Biotechnol J 15 (11):1371-1373. doi:10.1111/pbi.12788
Ma X N, Zhang X Y, Liu H M, Li Z H (2020) Highly efficient DNA-free plant genome editing using virally delivered CRISPR-Cas9. Nat Plants 6 (7):773-+. doi:10.1038/s41477-020-0704-5
Manghwar H, Lindsey K, Zhang X, Jin S (2019) CRISPR/Cas System: Recent Advances and Future Prospects for Genome Editing. Trends Plant Sci 24 (12):1102-1125. doi:10.1016/j.tplants.2019.09.006
Nasti R A, Voytas D F (2021) Attaining the promise of plant gene editing at scale. P Natl Acad Sci USA 118 (22). doi:ARTN e200484611710.1073/pnas.2004846117
Nguyen C, Li J, Mou B, Gong H, Huo H (2021) A case study of using an efficient CRISPR/Cas9 system to develop variegated lettuce. Vegetable Research 1 (1):1-10
Nisa M, Bergis C, Pedroza-Garcia J A, Drouin-Wahbi J, Mazubert C, Bergounioux C, Benhamed M, Raynaud C (2021) The plant DNA polymerase theta is essential for the repair of replication-associated DNA damage. Plant Journal 106 (5):1197-1207. doi:10.1111/tpj.15295
Nishihama R, Ishida S, Urawa H, Kamei Y, Kohchi T (2016) Conditional Gene Expression/Deletion Systems for Marchantia polymorpha Using its Own Heat-Shock Promoter and Cre/loxP-Mediated Site-Specific Recombination. Plant Cell Physiol 57 (2):271-280. doi:10.1093/pcp/pcv102
Nishizawa-Yokoi A, Saika H, Hara N, Lee L Y, Toki S, Gelvin S B (2021) Agrobacterium T-DNA integration in somatic cells does not require the activity of DNA polymerase theta. New Phytol 229 (5):2859-2872. doi:10.1111/nph.17032
Oh Y, Kim H, Kim S G (2021) Virus-induced plant genome editing. Current Opinion in Plant Biology 60. doi:ARTN 10199210.1016/j.pbi.2020.101992
Ozyigit I I, Kurtoglu K Y (2020) Particle bombardment technology and its applications in plants. Mol Biol Rep 47 (12):9831-9847. doi:10.1007/s11033-020-06001-5
Park J, Choe S (2019) DNA-free genome editing with preassembled CRISPR/Cas9 ribonucleoproteins in plants. Transgenic Res 28:61-64. doi:10.1007/s11248-019-00136-3
Pfaffl M W (2001) A new mathematical model for relative quantification in real-time R T-PCR. Nucleic Acids Res 29 (9). doi:ARTN e45 DOI 10.1093/nar/29.9.e45
Pramanik D, Shelake R M, Kim M J, Kim J Y (2021) CRISPR-Mediated Engineering across the Central Dogma in Plant Biology for Basic Research and Crop Improvement. Mol Plant 14 (1):127-150. doi:10.1016/j.molp.2020.11.002
Ramsden D A, Carvajal-Garcia J, Gupta G P (2021) Mechanism, cellular functions and cancer roles of polymerase-theta-mediated DNA end joining. Nat Rev Mol Cell Biol. doi:10.1038/s41580-021-00405-2
Saito S, Maeda R, Adachi N (2017) Dual loss of human POLQ and LIG4 abolishes random integration. Nat Commun 8. doi:ARTN 1611210.1038/ncomms16112
Sattar M N, Iqbal Z, Al-Khayri J M, Jain S M (2021) Induced Genetic Variations in Fruit Trees Using New Breeding Tools: Food Security and Climate Resilience. Plants-Basel 10 (7). doi:ARTN 134710.3390/plants10071347
Shen H, Strunks G D, Klemann BJPM, Hooykaas PJJ, de Pater S (2017) CRISPR/Cas9-Induced Double-Strand Break Repair in Arabidopsis Nonhomologous End-Joining Mutants. G3-Genes Genom Genet 7 (1):193-202. doi:10.1534/g3.116.035204
Sizova I, Kelterborn S, Verbenko V, Kateriya S, Hegemann P (2021) Chlamydomonas POLO is necessary for CRISPR/Cas9-mediated gene targeting. G3 (Bethesda). doi:10.1093/g3journal/jkab114
Tzfira T, Li J, Lacroix B, Citovsky V (2004) Agrobacterium T-DNA integration: molecules and models. Trends Genet 20 (8):375-383. doi:10.1016/j.tig.2004.06.004
Uranga M, Aragones V, Selma S, Vazquez-Vilar M, Orzaez D, Daros J A (2021) Efficient Cas9 multiplex editing using unspaced sgRNA arrays engineering in a Potato virus X vector. Plant Journal 106 (2):555-565. doi:10.1111/tpj.15164
van Kregten M, de Pater S, Romeijn R, van Schendel R, Hooykaas PJJ, Tijsterman M (2016) T-DNA integration in plants results from polymerase-theta-mediated DNA repair. Nat Plants 2 (11). doi:Artn 1616410.1038/Nplants.2016.164
van Tol N, van Schendel R, Bos A, van Kregten M, de Pater S, Hooykaas PJJ, Tijsterman M (2021) Gene targeting in Polymerase Theta-deficient Arabidopsis thaliana. Plant J. doi:10.1111/tpj.15557
Vu T V, Doan DTH, Kim J, Sung Y W, Tran M T, Song Y J, Das S, Kim J Y (2021) CRISPR/Cas-based precision genome editing via microhomology-mediated end joining. Plant Biotechnol J 19 (2):230-239. doi:10.1111/pbi.1349Woo J W, Kim J, Kwon S I, Corvalan C, Cho S W, Kim H, Kim S G, Kim S T, Choe S, Kim J S (2015) DNA-free genome editing in plants with preassembled CRISPR-Cas9 ribonucleoproteins. Nat Biotechnol 33 (11):1162-+. doi:10.1038/nbt.3389
Wu S, Zhu H, Liu J, Yang Q, Shao X, Bi F, Hu C, Huo H, Chen K, Yi G (2020) Establishment of a PEG-mediated protoplast transformation system based on DNA and CRISPR/Cas9 ribonucleoprotein complexes for banana. BMC Plant Biol 20 (1):425. doi:10.1186/s12870-020-02609-8
Yan P, Shen W T, Gao X Z, Li X Y, Zhou P, Duan J (2012) High-Throughput Construction of Intron-Containing Hairpin RNA Vectors for RNAi in Plants. Plos One 7 (5). doi:ARTN e38186 10.1371/journal.pone.0038186
Yang L T, Ding J Y, Zhang C M, Jia J W, Weng H B, Liu W X, Zhang D B (2005) Estimating the copy number of transgenes in transformed rice by real-time quantitative PCR. Plant Cell Rep 23 (10-11):759-763. doi:10.1007/s00299-004-0881-0
Zelensky A N, Schimmel J, Kool H, Kanaar R, Tijsterman M (2017) Inactivation of Pol theta and C-NHEJ eliminates off-target integration of exogenous DNA. Nat Commun 8. doi:ARTN 66 10.1038/s41467-017-00124-3
Zhang Y, Iaffaldano B, Qi Y (2021) CRISPR ribonucleoprotein-mediated genetic engineering in plants. Plant Commun 2 (2):100168. doi:10.1016/j.xplc.2021.100168
Zhang Y, Liang Z, Zong Y, Wang Y P, Liu J X, Chen K L, Qiu J L, Gao C X (2016) Efficient and transgene-free genome editing in wheat through transient expression of CRISPR/Cas9 DNA or RNA. Nat Commun 7. doi:ARTN 12617 10.1038/ncomms12617
Zhu H, Li C, Gao C (2020) Applications of CRISPR-Cas in agriculture and plant biotechnology. Nat Rev Mol Cell Biol 21 (11):661-677. doi:10.1038/s41580-020-00288-9

1. A method for editing a genome of a plant cell, the method comprising contacting the plant cell with an Agrobacterium that comprises a first nucleic acid sequence that encodes an oligonucleotide for silencing POLQ in the plant cell and a second nucleic acid sequence that encodes a genome editing system for editing a gene of interest, wherein contacting is conducted under conditions that allow for the first and second nucleic acid sequences to be introduced into the plant cell and the genome is transformed without integration of T-DNA.

2. The method of claim 1, wherein the plant cell comprises a cell of a non-seeded perennial plant or an annual plant.

3. The method of claim 1, wherein the plant cell is in an explant, plant organ, or seed.

4. The method of claim 1, wherein the Agrobacterium comprises Agrobacterium tumefaciens.

5. The method of claim 1, wherein the Agrobacterium has been modified by introduction of a plasmid comprising SEQ ID NO 17-18 or a fragment thereof, or a sequence comprising at least 95% identity therewith, wherein the plasmid comprises the first nucleic acid sequence and optionally, the second nucleic acid sequence.

6. The method of claim 1, wherein the genome-editing system comprises a nuclease, wherein the nuclease introduces a single-strand DNA break or a double-strand DNA break.

7. The method of claim 3, wherein the genome-editing technique comprises a TALEN, a ZFN, meganuclease, or a CRISPR/Cas system.

8. The method of any of claim 1, wherein the genome editing system introduces a genetic modification into the gene of interest or regulatory element thereof.

9. The method of claim 1, wherein the conditions comprise incubating the plant cell in a first callus induction media, a second callus induction media, and a shoot induction media.

10. The method of claim 9, wherein the second callus induction media comprises kanamycin.

11. A method for editing a genome of a tobacco plant cell, the method comprising contacting the tobacco plant cell with an Agrobacterium that comprises a first nucleic acid sequence that encodes an oligonucleotide for silencing POLQ and a second nucleic acid sequence that encodes a genome editing system for editing a gene of interest, wherein contacting is conducted under conditions that allow for the first and second nucleic acid sequences to be introduced into the tobacco plant cell and the genome is transformed without integration of T-DNA.

12. The method of claim 11, wherein the tobacco plant cell is in an explant, plant organ, or seed.

13. The method of claim 11, wherein the Agrobacterium comprises Agrobacterium tumefaciens.

14. The method of claim 11, wherein the Agrobacterium has been modified by introduction of a plasmid comprising SEQ ID NO 17-18 or a fragment thereof, or a sequence comprising at least 95% identity therewith, wherein the plasmid comprises the first nucleic acid sequence and optionally, the second nucleic acid sequence.

15. The method of claim 11, wherein the genome-editing system comprises a nuclease, wherein the nuclease introduces a single-strand DNA break or a double-strand DNA break.

16. The method of claim 15, wherein the genome-editing technique comprises a TALEN, a ZFN, meganuclease, or a CRISPR/Cas system.

17. The method of claim 11, wherein the genome editing system introduces a genetic modification into the gene of interest or regulatory element thereof.

18. The method of claim 11, wherein the conditions comprise a first condition, a second condition, and a third condition.

19. The method of claim 18, wherein the first condition comprises incubating the tobacco plant cells in a first callus induction medium for 2 days under no light.

20. The method of claim 19, wherein the first callus induction medium comprises MS salts, 30 mg/L sucrose, 0.5-1.0 mg/L benzylaminopurine, and 0.1-0.75 mg/L NAA.

21. The method of claim 18, wherein the second condition comprises incubating the tobacco plant cells in a second callus induction medium for 7 to 10 days under light.

22. The method of claim 21, wherein the second callus induction medium comprises 100 mg/L Timentin and 60 mg/L kanamycin.

23. The method of claim 18, wherein the third conditions comprise incubating the tobacco plant cells in a shoot induction medium.

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)


SEQUENCE LISTING (18 sequences)

SEQ ID NO: 1 - DNA, 18 bp, synthetic construct
  misc_feature 1..18: note = Primer
  source 1..18: mol_type = other DNA
acaagttcagcgtgtccg

SEQ ID NO: 2 - DNA, 18 bp, synthetic construct
  misc_feature 1..18: note = Primer
  source 1..18: mol_type = other DNA
tcaccttgatgccgttct

SEQ ID NO: 3 - DNA, 21 bp, synthetic construct
  misc_feature 1..21: note = Primer
  source 1..21: mol_type = other DNA
gctgggaactttactgcctcg

SEQ ID NO: 4 - DNA, 20 bp, synthetic construct
  misc_feature 1..20: note = Primer
  source 1..20: mol_type = other DNA
gggtcgctcaatgtccaatg

SEQ ID NO: 5 - DNA, 41 bp, synthetic construct
  misc_feature 1..41: note = Primer
  source 1..41: mol_type = other DNA
taggtctcaggagctcggttttagagctagaaatagcaagt

SEQ ID NO: 6 - DNA, 37 bp, synthetic construct
  misc_feature 1..37: note = Primer
  source 1..37: mol_type = other DNA
cgggtctcactccaaagataagctgtgcaccagccgg

SEQ ID NO: 7 - DNA, 41 bp, synthetic construct
  misc_feature 1..41: note = Primer
  source 1..41: mol_type = other DNA
taggtctcaataagttagttttagagctagaaatagcaagt

SEQ ID NO: 8 - DNA, 38 bp, synthetic construct
  misc_feature 1..38: note = Primer
  source 1..38: mol_type = other DNA
accaggtctcaggagacaccggttaagagaaatctgac

SEQ ID NO: 9 - DNA, 38 bp, synthetic construct; identical to SEQ ID NO: 8
  misc_feature 1..38: note = Primer
  source 1..38: mol_type = other DNA

SEQ ID NO: 10 - DNA, 40 bp, synthetic construct
  misc_feature 1..40: note = Primer
  source 1..40: mol_type = other DNA
accaggtctcatcgtgcttatctgagcttcgactgatgta

SEQ ID NO: 11 - DNA, 32 bp, synthetic construct
  misc_feature 1..32: note = Primer
  source 1..32: mol_type = other DNA
cgactagtggctcgtatgttgtgtggaattgt

SEQ ID NO: 12 - DNA, 34 bp, synthetic construct
  misc_feature 1..34: note = Primer
  source 1..34: mol_type = other DNA
gcgtcgacccgatctagtaacatagatgacaccg

SEQ ID NO: 13 - DNA, 20 bp, synthetic construct
  misc_feature 1..20: note = Primer
  source 1..20: mol_type = other DNA
acaactcctaggcggtttca

SEQ ID NO: 14 - DNA, 20 bp, synthetic construct
  misc_feature 1..20: note = Primer
  source 1..20: mol_type = other DNA
gaatgatcttccctccgaaa

SEQ ID NO: 15 - DNA, 21 bp, synthetic construct
  misc_feature 1..21: note = Primer
  source 1..21: mol_type = other DNA
tggaggggaatacatcagtcg

SEQ ID NO: 16 - DNA, 22 bp, synthetic construct
  misc_feature 1..22: note = Primer
  source 1..22: mol_type = other DNA
ccaggtgtctcggattcagttg

SEQ ID NO: 17 - DNA, 1812 bp, synthetic construct
  misc_feature 1..1812: note = Vector
  source 1..1812: mol_type = other DNA
cgactagtagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagatacatcacaatcacacaaaactaacaaaagatcaaaagcaagttcttcactgttgatagcttatctgagcttcgactgatgtattcccctccatgacctcaaaagaatttagagctgcatcgtcagaattggtttttctgtttatattagactcgttttcactcgagatgcagcggattctctttgcatgtttattatccacatcaagaatggaaggactaccactccttttactagcaccctcactctgggctgaaggtaaacttaaactagctggcagttcactgcaatataatgacaagaagttggtagcaaactgcttaagttctgagtttctggtaacctgtgcagtaaccaagacatcttctccatgacctctattttcaacaacacgtgagtcaagggaaataacagacttctcctttgaagtttttgtttcaaaagttgttgaacgtgctttgaccaaagaagtactctctttattttcatcttttagatatgaaccaatttctaatgtcagatttctcttaaccggtgtgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggttccggaaccaattcgtaatcatggtcatagctgtttcctgtgtgaaattgttatccgctcacaattccacacaacatacgagccggaagcataaagtgtaaagcctggggtgcctaatgagtgagctaactgtcgacatc

SEQ ID NO: 18 - DNA, 3792 bp, synthetic construct
  misc_feature 1..3792: note = Vector
  source 1..3792: mol_type = other DNA
cgactagtaagctttcaacatgtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacgtcgagagttctcaacacaacatatacaaaacaaacgaatctcaagcaatcaagcattctacttctattgcagcaatttaaatcatttcttttaaagcaaaagcaattttctgaaaattttcaccatttacgaacgatagccagggcccggagacaccggttaagagaaatctgacattagaaattggttcatatctaaaagatgaaaataaagagagtacttctttggtcaaagcacgttcaacaacttttgaaacaaaaacttcaaaggagaagtctgttatttcccttgactcacgtgttgttgaaaatagaggtcatggagaagatgtcttggttactgcacaggttaccagaaactcagaacttaagcagtttgctaccaacttcttgtcattatattgcagtgaactgccagctagtttaagtttaccttcagcccagagtgagggtgctagtaaaaggagtggtagtccttccattcttgatgtggataataaacatgcaaagagaatccgctgcatctcgagtgaaaacgagtctaatataaacagaaaaaccaattctgacgatgcagctctaaattcttttgaggtcatggaggggaatacatcagtcgaagctcagataagcacgagcccttggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatacggccggtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatcgcgtggatccggcttactaaaagccagataacagtatgcgtatttgcgcgctgatttttgcggtataagaatatatactgatatgtcggtcccataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagttggtctagagatttcgtctagatcgtgcttatctgagcttcgactgatgtattcccctccatgacctcaaaagaatttagagctgcatcgtcagaattggtttttctgtttatattagactcgttttcactcgagatgcagcggattctctttgcatgtttattatccacatcaagaatggaaggactaccactccttttactagcaccctcactctgggctgaaggtaaacttaaactagctggcagttcactgcaatataatgacaagaagttggtagcaaactgcttaagttctgagtttctggtaacctgtgcagtaaccaagacatcttctccatgacctctattttcaacaacacgtgagtcaagggaaataacagacttctcctttgaagtttttgtttcaaaagttgttgaacgtgctttgaccaaagaagtactctctttattttcatcttttagatatgaaccaatttctaatgtcagatttctcttaaccggtgtctccgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggtcgacgc